(19) United States
(12) Patent Application Publication
(10) Pub. No.: US 2011/0082519 A1
(43) Pub. Date: Apr. 7, 2011

(54) Hearing Implant Fitting

(75) Inventors: Stefan Strahl, Innsbruck (AT); Philipp Spitzer, Innsbruck (AT)

(73) Assignee: MED-EL ELEKTROMEDIZINISCHE GERAETE GMBH, Innsbruck (AT)

(21) Appl. No.: 12/890,188

(22) Filed: Sep. 24, 2010

Related U.S. Application Data
(60) Provisional application No. 61/245,887, filed on Sep. 25, 2009.

(51) Int. Cl. A61N 1/36

(52) U.S. Cl. 607/57

(57) ABSTRACT

A system and method of fitting a cochlear implant of a patient includes analyzing data of one or more previously fitted cochlear implant users. Predicted fitting data for the patient is provided based on the analysis. Stimulation parameters of the cochlear implant are adjusted based, at least in part, on the predicted fitting data. Further steps are suggested to minimize the prediction error.

### CROSS REFERENCE TO RELATED APPLICATIONS

This application claims priority from U.S. provisional patent application Ser. No. 61/245,887 filed Sep. 25, 2009, entitled Hearing Implant Fitting, the disclosure of which is hereby incorporated herein by reference in its entirety.

### TECHNICAL FIELD

The present invention relates to hearing implants or hearing aids, and more particularly to a system and method for fitting a hearing implant or hearing aid, such as a cochlear implant, to a patient.

### BACKGROUND ART

Cochlear implants and other inner ear prostheses are one option to help profoundly deaf or severely hearing impaired persons. Unlike conventional hearing aids that just apply an amplified and modified sound signal; a cochlear implant is based on direct electrical stimulation of the acoustic nerve. Typically, a cochlear implant stimulates neural structures in the inner ear electrically in such a way that hearing impressions most similar to normal hearing is obtained.

A normal ear transmits sounds as shown in FIG. 1 through the outer ear 101 to the tympanic membrane (eardrum) 102, which moves the bones of the middle ear 103 (malleus, incus, and stapes) that vibrate the oval window and round window openings of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

Some persons have partial or full loss of normal sensorineural hearing. Cochlear implant systems have been developed to overcome this by directly stimulating the user's cochlea 104. A typical cochlear prosthesis may include two parts: the speech processor 111 and the implanted stimulator 108. The speech processor 111 typically includes a microphone, a power supply (batteries) for the overall system and a processor that is used to perform signal processing of the acoustic signal to extract the stimulation parameters. The speech processor may be a behind-the-ear (BTE-) device.

The stimulator 108 generates the stimulation patterns (based on the extracted audio information) that is sent through an electrode lead 109 to an implanted electrode array 110. Typically, this electrode array 110 includes multiple electrodes on its surface that provide selective stimulation of the cochlea 104. For example, each electrode of the cochlear implant is often stimulated with signals within an assigned frequency band based on the organization of the inner ear. The placement of each electrode within the cochlea is typically based on its assigned frequency band, with electrodes closer to the base of the cochlea generally corresponding to higher frequency bands.

The connection between speech processor and stimulator is usually established by means of a radio frequency (RF-) link. Note that via the RF-link both stimulation energy and stimulation information are conveyed. Typically, digital data transfer protocols employing bit rates of some hundreds of kBit/s are used.

After implantation, a cochlear implant is adjusted for the patient. More particularly, using interactive software and computer hardware, an audiologist “fits” the cochlear implant to the patient by adjusting one or more parameters to improve hearing. The better the fitting, the better the performance of the hearing impaired patient.

Objective data and/or subjective data may be used in optimizing the fitting. For example, two of the most common subjective measurements used to adjust the cochlear implant include: a most comfort loudness level (MCL) which indicates the level at which sound is loud but comfortable; and a threshold level (THR) which indicates the softest input detected through the implant (both the MCL and THR are clinical measurements of current). The MCL and THR levels may be determined, in part, using verbal feedback from an adult patient or facial reactions from a small child. Examples of objective data used to fit a patient's cochlear implant include: the Electrical Stapedius Reflex Test (ESR-T); measurement of Electrically evoked Compound Action Potential (ECAP), and Brainstem Evoked Response Audiometry (BERA) testing. Objective data is particularly useful in determining fitting levels for very young children who are unable to provide important feedback about their listening experience. The overall fitting process may last weeks or even months and has to be adjusted at regular intervals (e.g. once per year).

### SUMMARY OF THE INVENTION

In accordance with an embodiment of the invention, a method of fitting a cochlear implant of a patient includes analyzing data of one or more previously fitted cochlear implant users. Predicted fitting data for the patient is provided based on the analysis. Stimulation parameters of the cochlear implant are adjusted based, at least in part, on the predicted fitting data.

In accordance with related embodiments of the invention, the data may include test results and/or fitting data from a previously fitted cochlear implant user. The fitting data of the previously fitted cochlear implant user may include MCL and/or THR. A database of previously fitted cochlear implant users may be provided and/or retained, from which the data is retrieved and analyzed. Analyzing the data of previously fitted cochlear implant users may include inputting data associated with the patient.

In accordance with further related embodiments of the invention, analyzing the data may include conducting statistical analysis on the data. The statistical analysis may include performing at least one of a mean, a distribution, a standard deviation, and a multiple regression analysis. At least one of a confidence interval and a probability distribution associated with the predicted fitting data may be provided. Analyzing the data may include providing a recording to test the patient, the method further including testing the patient with the recording, and recalculating the confidence level.

In accordance with another embodiment of the invention, a computer program product for fitting a cochlear implant of a patient is presented. The computer program product includes a computer usable medium having computer readable program code thereon. The computer readable program code includes program code for analyzing data of one or more previously fitted cochlear implant users; program code for providing predicted fitting data for the patient based on the analysis; and program code for displaying the predicted fitting data.

In accordance with related embodiments of the invention, the data may include test results and/or fitting data from a previously fitted cochlear implant user. The fitting data of the previously fitted cochlear implant user may include MCL and/or THR. A database of previously fitted cochlear implant users may be provided and/or retained, from which the data is retrieved and analyzed.

In accordance with further related embodiments of the invention, the program code for analyzing the data may include program code for conducting statistical analysis on the data. Conducting the statistical analysis may include program code for performing a mean, a standard deviation, and/or a multiple regression analysis. At least one of a confidence interval and a probability distribution associated with the predicted fitting data may be provided. The program code for analyzing the data may include program code for providing a recording to test the patient, and program code for recalculating the confidence level based, at least in part, on test results of the patient in response to the recording. The program code for analyzing the data of previously fitted cochlear implant users may include program code for inputting data associated with the patient.

In accordance with yet another embodiment of the invention, a system for fitting a cochlear implant of a patient includes a processor for analyzing data of previously fitted cochlear implant users and providing predicted fitting data for the patient. The system further includes a display for displaying the predicted fitting data.

In accordance with related embodiments of the invention, the data may include test results and/or fitting data from a previously fitted cochlear implant user. The fitting data of the previously fitted cochlear implant user may include MCL and/or THR. A database of previously fitted cochlear implant users may be provided and/or retained, from which the data is retrieved and analyzed. The database may include data associated with the patient.

In accordance with further related embodiments of the invention, the processor may conduct statistical analysis on the data. The processor may perform a mean, a standard deviation, and/or a multiple regression analysis on the data. The processor may provide at least one of a confidence interval and a probability distribution associated with the predicted fitting data. The processor may provide a recording to test the patient, and recalculate the confidence level based, at least in part, on test results of the patient in response to the recording.

In accordance with another embodiment of the invention, a method of fitting at least one of a hearing device of a patient is presented. The method includes analyzing data of one or more previously fitted hearing device users. Predicted fitting data for the patient based on the analysis is provided. Stimulation parameters of the hearing device are adjusted based, at least in part, on the predicted fitting data.

In accordance with related embodiments of the invention, the device is one of a cochlear implant, a middle ear implant, a hearing aid, an electro-acoustical stimulation implant, and an optical stimulation implant. The device may include a combination of various technologies as known in the art, such as, without limitation, electro-optical, opto-mechanical, opto-acoustical technology/devices.

In accordance with yet another embodiment of the invention, a computer program product for fitting a hearing device of a patient is presented. The computer program product includes a computer usable medium having computer readable program code thereon. The computer readable program code includes program code for analyzing data of one or more previously fitted hearing device users; program code for providing predicted fitting data for the patient based on the analysis; and program code for displaying the predicted fitting data.

In accordance with related embodiments of the invention, the device is one of a cochlear implant, a middle ear implant, a hearing aid, an electro-acoustical stimulation implant, and an optical stimulation implant.

In accordance with yet another embodiment of the invention, a system for fitting a hearing device of a patient includes a processor for analyzing data of one or more previously fitted hearing device users and providing predicted fitting data for the patient based on the analyzed data. A display displays the predicted fitting data.

In accordance with related embodiments of the invention, the device is one of a cochlear implant, a middle ear implant, a hearing aid, an electro-acoustical stimulation implant, and an optical stimulation implant. The device may include a combination of various technologies as known in the art, such as, without limitation, electro-optical, opto-mechanical, opto-acoustical technology/devices. The processor may be operatively coupled to a database that includes data from previously fitted cochlear implant users;

### BRIEF DESCRIPTION OF THE DRAWINGS

The foregoing features of the invention will be more readily understood by reference to the following detailed description, taken with reference to the accompanying drawings, in which:

FIG. 1 illustrates a sectional view of an ear connected to a cochlear implant system, in accordance with an embodiment of the invention;

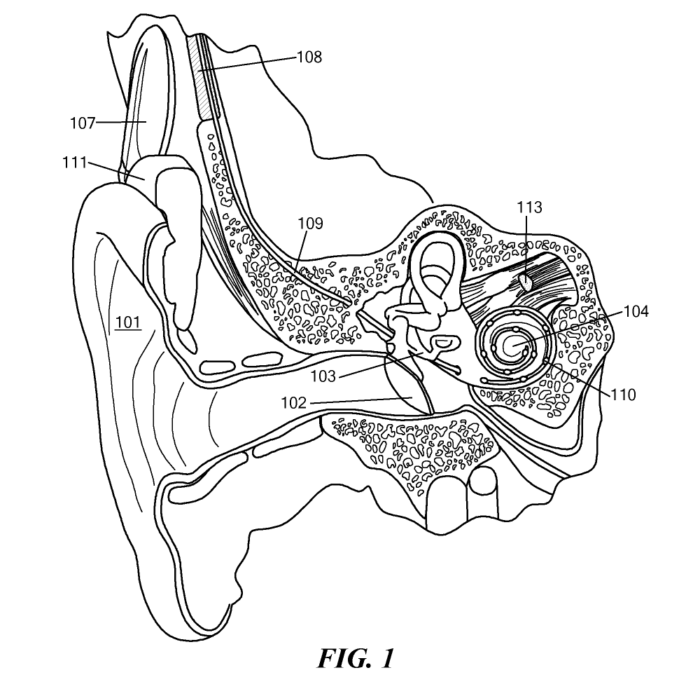

FIG. 2 shows an exemplary graphic display, in accordance with an embodiment of the invention;

FIG. 3 shows an exemplary graphic display for fine-tuning prediction options, in accordance with an embodiment of the invention;

FIG. 4 shows an exemplary graphic display depicting the history of single or multiple electrodes, in accordance with an embodiment of the invention; and

FIG. 5 shows an exemplary graphic display depicting recording selections for use in obtaining the prediction, in accordance with an embodiment of the invention.

### DETAILED DESCRIPTION OF SPECIFIC EMBODIMENTS

In illustrative embodiments of the invention, a system and method of fitting a hearing device of a patient, such as cochlear implant, includes performing analysis on a database that includes data from one or more previous cochlear implant users. Automatic calculation of the most probable correct final fitting value may be provided based, in part, on the analysis. In this manner, the complexity and/or time needed to fit the cochlear implant may advantageously be reduced. Details are discussed below.

While a system and method of fitting a hearing device of a patient is illustratively described below with reference to a cochlear implant, embodiments of the invention may pertain to other hearing devices. For example and without limitation, the device may be a middle ear implant, a conventional hearing aid, an electro-acoustical stimulation implant, or an optical stimulation implant.

Table 1 shows various data from previous cochlear implant users that may be analyzed in fitting a cochlear implant of a patient, in accordance with an embodiment of the invention. The previous cochlear implant user associated with the data may be presented in an anonymous manner in the database. In addition to data associated with previous cochlear implant users, the data base may be supplemented with the (current) patient data when known. The data may be provided, without limitation, in a database. The analysis may be performed using, without limitation, a computer or processor, that may be operatively coupled to the database and/or an appropriate display as known in the art.

There may be one or more databases that are provided, for example, to an audiologist, along with fitting-software and additionally, the database from the clinic itself, that increases as time goes by. In various embodiments, the quality of database entries may be rated either manually or automatically, allowing usage of, for example, a fuzzy algorithm for deciding the next suggested measurement.

Referring to Table 1, the previous cochlear implant user and/or patient related input data and associated subjective and objective test results with it error estimations are called “input data.” The parameters of the final fitting for the previous cochlear implant user and/or patient, for example the MCL and THR for a predefined coding strategy, are called “fitting-data.”

TABLE 1

| | |
|---|---|
| Birthday | Input data |
| Date of surgery | |
| Type of implant | |
| Type of electrode array | |
| . . . | |
| Kind of disease that lead to the CI | |
| Date/progression time frame of hearing loss | |
| Preoperative Audiogram | |
| Number of inserted electrode contacts | |
| Position of the electrode within the cochlear | |
| (if available from e.g. X-Ray) | |
| . . . | |
| ART(auditory nerve response telemetry)-results | |
| (intra/postoperativly) | |
| ESR-T (evoked stapedius reflex threshold) results | |
| (intra/postoperativly) | |
| EBERA results | |
| . . . | |
| Loudness judgement of defined sequences | |
| THR determination of defined sequences | |
| Behavioural reaction (for uncooperative patients, | |
| e.g. children) due to a defined sequence (no | |
| reaction, small reaction, strong reaction ...) | |
| . . . | |
| Preferred final fitting(s): | fitting-data |
| Coding-strategy | |
| Used MCL of each electrode | |
| Used THR of each electrode | |
| Rate | |
| Speech processor settings | |
| Frequency-Electrode mapping | |
| . . . | |

Having the database in the background, a statistical analysis may be performed to predict the most probable fitting-data (e.g., most probable MCL and THR values) for the patient. In various embodiments, a distribution and/or confidence interval may advantageously be calculated for the predicted fitting-data. Generally, increasing the amount of patient-input data (such as, without limitation, behavioural data, ESR-T data, ART data, time course of a data source, and/or type of disease) in the database results in a better prediction.

The prediction of the most probable fitting-data for the patient, which may be provided with confidence intervals, may be provided to an audiologist. For example, the predication may be visualized/shown on a display.

For the simple case that no patient-input data is available, histograms of the fitting-data of one or more previous cochlear implant users in the database may be provided, with, for example, the mean value and standard deviation.

If patient-input parameters (such as, without limitation, age, ESR-T thresholds, and/or behavioural test results) are known, an analysis may be performed in providing the most probable fitting-fitting data, using both the patient-input parameters and the data associated with the previous cochlear implant patients. For example, a multiple regression analysis may be calculated (with the patient-input as predictor variables and the fitting-data as criterion variables) to predict the fitting-data for the patient including, without limitation, the confidence intervals. A non-parametric regression technique that automatically models non-linearities and interaction may be used for calculating the multivariate regression analysis if no general relationship between some patient-input and fitting-data are known, for example, from literature.

In various embodiments, a suggested recording may also be provided to the audiologist, which can then be used to further test the patient, so as to achieve even a better prediction. For example, the suggested recording may be determined based, at least in part, on the informative, objective and/or subjective data to effectively reduce the prediction error (e.g., width of the confidence intervals). Safe values for suggested recordings may also be determined.

Illustratively, for each possible patient-input parameter (such as subjective test results, objective test results and/or other information), a simulated recording may be performed. The result could be, without limitation, a random value taking the current probability distribution for the simulated patient-input parameter into account. For each simulated recording, new confidence intervals may be calculated and the patient-input parameter having the largest effect in optimizing the confidence levels may be recommended as the next test. In various embodiments, the audiologist is free to perform whatever test is desired, regardless of the suggested recording. If the quality of the data base is known, an algorithm such as, without limitation, a fuzzy algorithm, may be used to select the database that achieves the highest quality in the prediction of the new confidence interval.

In doing the above-described analysis/testing, psychophysically determined levels may be advantageously separated from fitting levels. For uncooperative patient (e.g., small children), visible reactions vs. no visible reactions is a test result that may be used. Each result may be “accepted” or “declined,” and if the result is accepted an error estimation may be provided (this is also true for psychophysically determined levels like MCL).

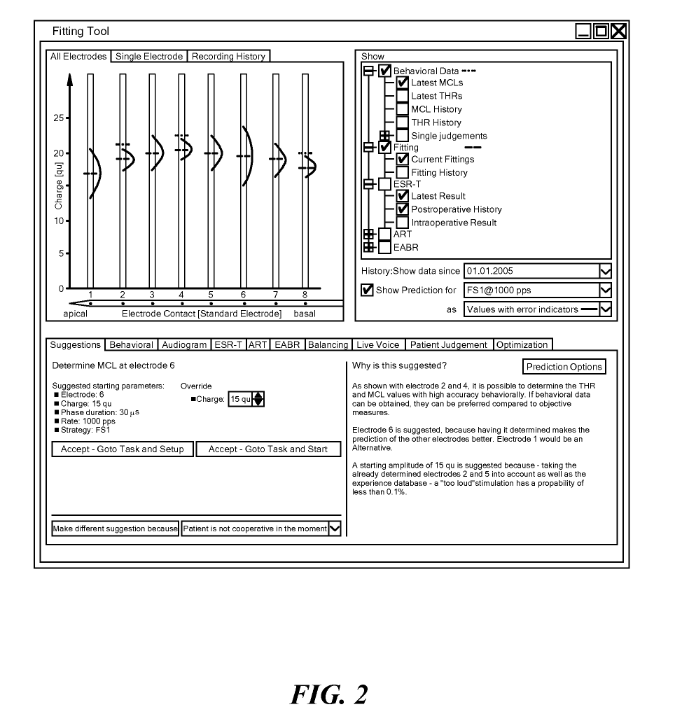

FIG. 2 shows an exemplary graphic display, in accordance with an embodiment of the invention. The curves depicting the predicted distributions are based, at least in part, from the previous recordings and the databases in the background. Also shown are lines depicting the last used fitting values, and lines showing the psychophysically determined MCL values. The audiologist may select what is shown in the graphic display by selecting various items in the box on the top-right side of the graphic display.

In the bottom part of the screen shown in FIG. 2, the suggested recording to be optionally used to further test the patient is shown. The audiologist may select “Accept—Goto Task and Setup” which would provide the option to fine-tune the suggestion(s). Alternatively, the audiologist may select “Accept—Goto Task and Start” to immediately start the recording in the corresponding task. Values that the audiologist may want to change often may be changed above the accept button to save time. There is also an explanation on the right hand side of the screen of FIG. 2, explaining why the recording was suggested to the audiologist.

If the audiologist does not wish to perform the suggested recording, the audiologist may select, in the bottom right of the screen shown in FIG. 2 the reason why. Furthermore, the audiologist may click on “Make different suggestion,” whereupon the system suggests an alternative that would also increase the prediction accuracy as much as possible. Alternatively, the audiologist has the option to perform whatever recording or task is desired. The results may then be incorporated in the next prediction. This is indicated by the tabs in the screenshot.

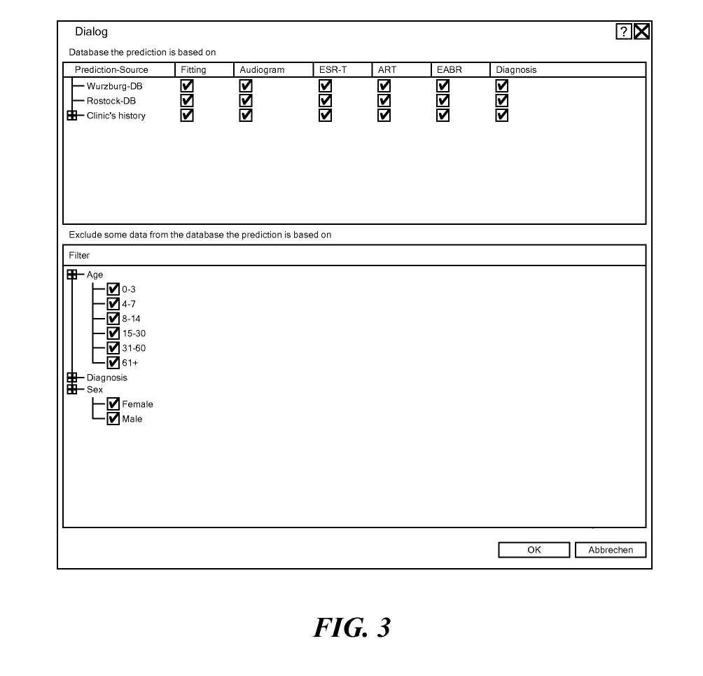

FIG. 3 shows an exemplary graphic display for fine-tuning prediction options, in accordance with an embodiment of the invention. The prediction options would allow the audiologist the capability to fine-tune what data in the database is used in making the predictions (e.g., the most probable fitting-fitting data and/or the suggested next test). For example, data from specific patients in the database may be excluded, or patients of a specific age or sex. More advanced options may be presented, for example, with an SQL-like language. In various embodiments, analysis may include determining “bad” and “good” correlations of various data in the database, so as enhance the prediction. Further, any analysis tool used may have the capability to learn from previous results so as to enhance the prediction.

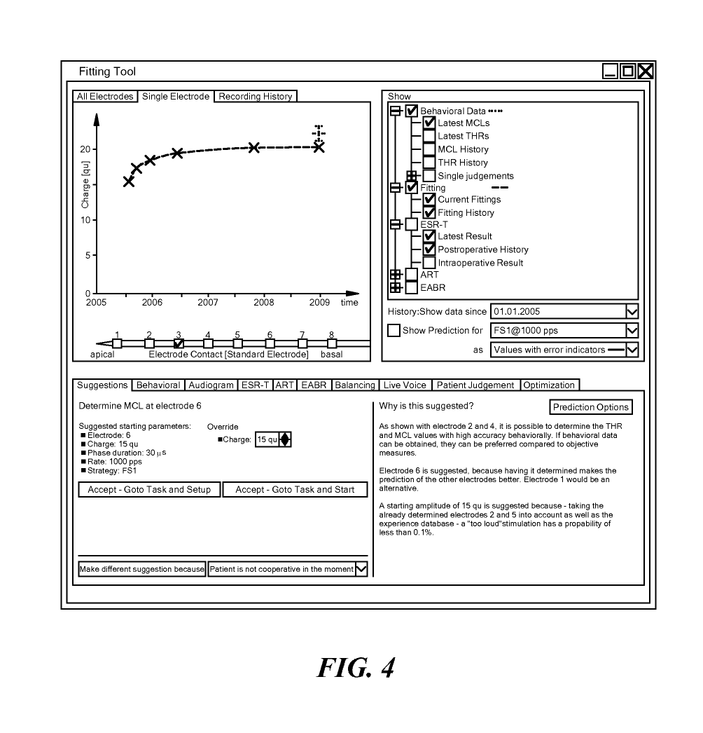

FIG. 4 shows an exemplary graphic display depicting the history of single or multiple electrodes, in accordance with an embodiment of the invention. In particular, FIG. 4 shows, without limitation, the selected history of electrode three with respect to year and charge. Other data associated with the electrode(s) may also be shown.

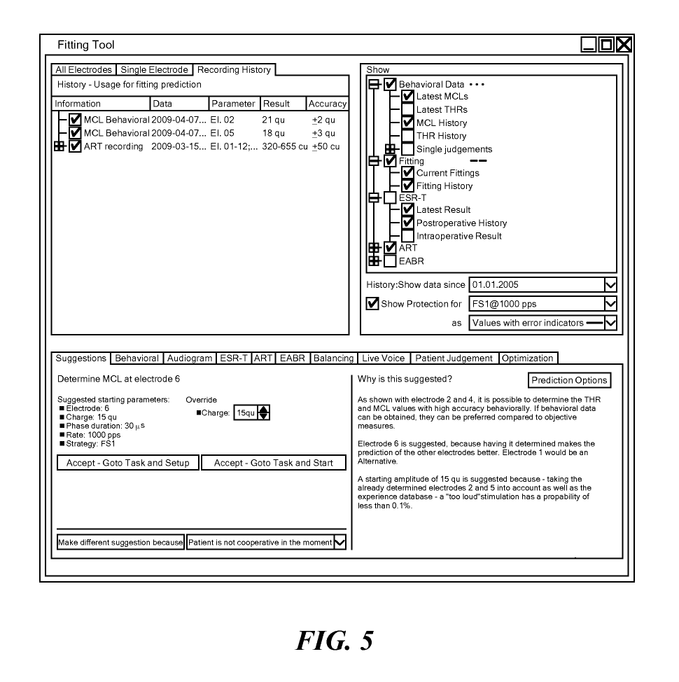

In various embodiments, the system keeps track of which recordings are good or bad. Each type of information may have an “accept” and “decline” option and in case of acceptance, error estimation should be given. FIG. 5 shows an exemplary graphic display depicting recording selections for use in obtaining the prediction, in accordance with an embodiment of the invention.

Embodiments of the invention may be implemented in whole or in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments can be implemented in whole or in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

An exemplary pseudo code representation (e.g., using Python programming language) of one specific approach of the above-described embodiments is depicted in Table 2 below, in accordance with an embodiment of the invention.

```
TABLE 2
# Main (pseudo) program
# ---------------------
# Load database(s) and store it in the “patients” variable
patients = load_database(‘shipped_database.db’)
# The current to-fit patient is created here.
# Alternatively, her/his already acquired data may also be loaded.
# This variable may includes all already acquired recording results.
patient = create_new_patient( )
# Start an infinite loop
while True:
    # predicted (mean and standard deviation) value of MCL (maximum
    # comfortable loudness)for electrode 1, 2, 3 (to keep it short in
    # this example)
    # The predictor function “predict_mcl” is defined elsewhere (see
    # below)
    pre_mean_mcl_el01,
    pre_stddev_mcl_el01 = predict_mcl(patients, patient, 1)
    pre_mean_mcl_el02,
    pre_stddev_mcl_el02 = predict_mcl(patients, patient, 2)
    pre_mean_mcl_el03,
    pre_stddev_mcl_el03 = predict_mcl(patients, patient, 3)
    # Show the predicted values
    plot_prediction(pre_mean_mcl_el01, pre_stddev_mcl_el01, 1)
    plot_prediction(pre_mean_mcl_el02, pre_stddev_mcl_el02, 2)
    plot_prediction(pre_mean_mcl_el03, pre_stddev_mcl_el03, 3)
    # Show already acquired results (none at the first iteration)
    plot_results(patient)
    # Suggest new recording to the audiologist and ask him/her what to
    do
    # The suggest_recording method is defined elsewhere (see below)
    suggested_recording = suggest_recording(patients, patient)
    choice = ask_user(suggested_recording)
    if choice == ‘abort’:
        # leve while loop and exit
        break
    elif choice == ‘ecap_el01’:
        # Perform ECAP recording on electrode 1
        patient.add_recording(perform_ecap(1))
    elif choice == ‘ecap_el02’:
        # Perform ECAP recording on electrode 2
        patient.add_recording(perform_ecap(2))
    elif choice == ‘esrt_el01’:
        # Perform ESRT recording on electrode 1
        patient.add_recording(perform_esrt(1))
    # ... there are many other possible recordings.
# Predictor function for MCL (maximum comfortable loudness)
# ------------------------------------------------------------------------
# This implemenation filters the database for people with similar results
# as the to-fit patient to predict the mcl values based on the remaining
# entries.
def predict_mcl(patients, patient, electrode):
```

| | |
|---|---|
| # patients | ... the database of all patients to use |
| # patient | ... the recording results of the to-fit patient |
| # electrode | ... electrode contact number for the prediction, e.g. 2. |

```
    # Filter database for sex:
    if patient.sex_is_available( ):
        patients = filter_sex(patients, patient.sex( ))
    # Filter database for similar ECAP threshold value of electrode 1
    if patient.ecap_threshold_available(electrode = 1):
        patients=filter_ecap(patients,
        mean(patient.ecap_threshold(electrode=1)))
    # Filter database for similar ECAP threshold value of electrode 2
    if patient.ecap_threshold_available(electrode = 2):
        patients=filter_ecap(patients,
        mean(patient.ecap_threshold(electrode=2)))
    # Filter database for similar ESRT (evoked stapedius reflex threshold)
    # value
    if patient.esrt_threshold_available(electrode = 1):
        patients=filter_esrt(patients,
        mean(patient.esrt_threshold(electrode=1)))
    # ... other filter possibilities are skipped here
    # return mean value and standard deviation of the remaining patients
    # in the database
    return mean(patients.mcl(electrode)), stddev(patients.mcl(electrode))
# Suggest_recording function
# ---------------------------------------------------------
#
def suggest_recording(patients, patient):
    best_recording = None
    smallest_sum_stddev_mcl=inf # smallest standard deviation of the
sum of all MCLs (infinite)
    # Loop over all undone recordings
    for possible_recording in patients.undone_recordings( ):
        p = copy(patient)
        # The simulate_recording function predicts a recording result
        # based on the database like the predict_mcl function predicts
        # the MCL value based on the database.
        p.add_recording(simulate_recordng(patients, patient))
        # Calculate the new sum of standard deviations of the MCL
        # by iterating over all electrodes
        sum_stddev_mcl = 0
        for electrode in electrodes:
            p_mean_mcl, p_stddev_mcl =
            predict_mcl(patients, p, electrode)
            sum_stddev_mcl = sum_stddev_mcl + p_stddev_mcl
        # Compare it with the best simulated result from the past
        if sum_stddev_mcl < smallest_sum_stddev_mcl:
            best_recording = possible_recording
            smallest_sum_stddev_mcl = sum_stddev_mcl
    # Return the best suggestion
    return best_recording
```

Although various exemplary embodiments of the invention have been disclosed, it should be apparent to those skilled in the art that various changes and modifications can be made which will achieve some of the advantages of the invention without departing from the true scope of the invention as defined in the appended claims.

What is claimed is:

1. A method of fitting a cochlear implant of a patient, the method comprising:
analyzing data of one or more previously fitted cochlear implant users;
providing predicted fitting data for the patient based on the analysis; and
adjusting stimulation parameters of the cochlear implant based, at least in part, on the predicted fitting data.

2. The method according to claim 1, wherein the data includes test results from a previously fitted cochlear implant user.

3. The method according to claim 1, wherein the data includes fitting data of a previously fitted cochlear implant user.

4. The method according to claim 1, wherein analyzing the data includes conducting statistical analysis on the data.

5. The method according to claim 1, further comprising providing at least one of a confidence interval and a probability distribution associated with the predicted fitting data.

6. The method according to claim 5, wherein analyzing the data includes providing a recording to test the patient, the method further comprising:
testing the patient with the recording; and
recalculating the confidence level.

7. The method according to claim 1, further comprising retaining a database of previously fitted cochlear implant users, from which the data is retrieved and analyzed.

8. A computer program product for fitting a cochlear implant of a patient, the computer program product comprising a computer usable medium having computer readable program code thereon, the computer readable program code comprising:
program code for analyzing data of one or more previously fitted cochlear implant users; and
program code for providing predicted fitting data for the patient based on the analysis; and
program code for displaying the predicted fitting data.

9. The computer program product according to claim 8, wherein the data includes test results from a previously fitted cochlear implant user.

10. The computer program product according to claim 8, wherein the data includes fitting data of a previously fitted cochlear implant user.

11. The computer program product according to claim 8, wherein the program code for analyzing the data includes program code for conducting statistical analysis on the data.

12. The computer program product according to claim 8, further comprising program code for providing at least one of a confidence interval and a probability distribution associated with the predicted fitting data.

13. The computer program product according to claim 12, wherein the program code for analyzing the data includes program code for providing a recording to test the patient, the computer readable program code further comprising:
program code for recalculating the confidence level based, at least in part, on test results of the patient in response to the recording.

14. The computer program product according to claim 8, further comprising program code for retaining a database of previously fitted cochlear implant users, from which the data is retrieved and analyzed.

15. A system for fitting a cochlear implant of a patient, the system comprising:
a processor for analyzing data of one or more previously fitted cochlear implant users and providing predicted fitting data for the patient; and
a display for displaying the predicted fitting data.

16. The system according to claim 15, wherein the data includes test results from a previously fitted cochlear implant user.

17. The system according to claim 15, wherein the data includes fitting data of a previously fitted cochlear implant user.

18. The system according to claim 21, wherein the processor conducts statistical analysis on the data.

19. The system according to claim 15, wherein the processor provides at least one of a confidence interval and a probability distribution associated with the predicted fitting data.

20. The system according to claim 19, wherein the processor provides a recording to test the patient, and wherein the processor recalculates the confidence level based, at least in part, on test results of the patient in response to the recording.

21. The system according to claim 21, further comprising a database of previously fitted cochlear implant users, from which the data is retrieved and analyzed.

22. A method of fitting at least one of a hearing device of a patient, the method comprising:
analyzing data of one or more previously fitted hearing device users; and
providing predicted fitting data for the patient based on the analysis; and
adjusting stimulation parameters of the hearing device based, at least in part, on the predicted fitting data.

23. The method according to claim 22, wherein the device is one of a cochlear implant, a middle ear implant, a hearing aid, an electro-acoustical stimulation implant, and an optical stimulation implant.

24. A computer program product for fitting a hearing device of a patient, the computer program product comprising a computer usable medium having computer readable program code thereon, the computer readable program code comprising:
program code for analyzing data of one or more previously fitted hearing device users; and
program code for providing predicted fitting data for the patient based on the analysis; and
program code for displaying the predicted fitting data.

25. The computer program product according to claim 24, wherein the device is one of a cochlear implant, a middle ear implant, a hearing aid, an electro-acoustical stimulation implant, and an optical stimulation implant.

26. A system for fitting a hearing device of a patient, the system comprising:
a database that includes data from previously fitted cochlear implant users;
a processor operatively coupled to the database for analyzing data of one or more previously fitted hearing device users and providing predicted fitting data for the patient;
a display for displaying the predicted fitting data.

27. The system according to claim 26, wherein the device is one of a cochlear implant, a middle ear implant, a hearing aid, an electro-acoustical stimulation implant, and an optical stimulation implant.
